(19) United States
(12) Patent Application Publication
(10) Pub. No.: US 2010/0286274 A1
(43) Pub. Date: Nov. 11, 2010

(54) Methods of Diagnosing, Monitoring and Treating Pulmonary Diseases

(75) Inventors: Amir Pelleg, Haverford, PA (US); Peter J. Barnes, London (GB); Sergei A. Kharitonov, London (GB)

Correspondence Address: FISH & RICHARDSON P.C., P.O. BOX 1022, MINNEAPOLIS, MN, 55440-1022, US

(73) Assignee: DUSKA SCIENTIFIC CO., Philadelphia, PA (US)

(21) Appl. No.: 12/839,196

(22) Filed: Jul. 19, 2010

Related U.S. Application Data
(62) Division of application No. 11/187,753, filed on Jul. 22, 2005.
(60) Provisional application No. 60/662,033, filed on Mar. 15, 2005.
(60) Provisional application No. 60/590,101, filed on Jul. 22, 2004.

(51) Int. Cl. A61K 31/196; A61P 11/00; A61P 11/14; A61P 11/08; A61P 11/06

(52) U.S. Cl. 514/563

(57) ABSTRACT

The invention includes methods of discriminating asthma from obstructive pulmonary disease, of treating pulmonary diseases, of treating cough, of assessing the efficacy of a treatment for an obstructive pulmonary disease, and of inhibiting activation of a P2-purinoreceptor (P2R).

This application claims the benefit of prior U.S. Provisional Application No. 60/590,101, filed Jul. 22, 2004, and prior U.S. Provisional Application No. 60/662,033, filed Mar. 15, 2005. The disclosures of U.S. Provisional Applications 60/590,101 and 60/662,033 are incorporated herein by reference in their entirety.

### TECHNICAL FIELD

This invention relates to pulmonary diseases, and more particularly to the treatment of pulmonary diseases (e.g., cough or obstructive pulmonary disease), and to the diagnosis, monitoring, and treatment of pulmonary diseases such as asthma and chronic obstructive pulmonary disease.

### BACKGROUND

Pulmonary diseases such as obstructive pulmonary disease (OPD) and cough continue to be both medically and economically devastating. For example, chronic obstructive pulmonary disease (COPD) is currently the fourth leading cause of death in the U.S. and is expected to be the third in the year 2020. An estimated 10 million adult Americans have COPD, and the prevalence is rising. Direct and indirect costs of managing COPD exceed $32 billion annually [Mapel (2004). Manag. Care Interface 17:61-6]. The World Health Organization (WHO) estimated that 2.74 deaths worldwide were caused by COPD in the year 2000 [Burney P. (2003) Eur. Respir. J. Suppl. 43:1s-44s]. Thus, there is an urgent need to develop methods to diagnose, monitor, and treat COPD, other OPD, and cough.

### SUMMARY

The invention is based in part on the discovery that (i) adenosine 5′-triphosphate (ATP) and related compounds activate vagal sensory nerve terminals associated with OPD, symptoms of OPD, or cough and (ii) such activation can be effectively inhibited with certain P2-purinoreceptor (P2R) antagonists. These findings provide the basis for monitoring the efficacy of treatment for OPD and for a test to distinguish asthma from COPD. In addition, the present invention features methods for treating OPD and cough.

More specifically, the invention provides a method of diagnosis. The method includes: (a) identifying a test subject suspected of having asthma or a chronic pulmonary obstructive disease (COPD); (b) administering a provocator compound to the subject; (c) determining a difference in lung function between before and after the administration; (d) determining whether the difference in lung function more closely resembles the difference in the lung function in control subjects having (i) asthma or (ii) COPD; and (e) classifying the test subject as: (1) likely to have asthma if the difference in lung function in the test subject more closely resembles the difference in lung function in control subjects having asthma than the difference in lung function in control subjects having COPD; or (2) likely to have COPD if the difference in lung function in the test subject more closely resembles the difference in lung function in control subjects having COPD than the difference in lung function in control subjects having asthma. The change in lung function can be determined, for example, as a function of the amount of the provocator compound that is required to cause an arbitrary particular change in forced expiratory volume (FEV₁), specific airway conductance (sGaw), Borg score, functional residual capacity (FRC), forced expiratory flow (FEF), and peak expiratory flow rate (PEFR). The arbitrary particular change can be a decrease or increase of greater than about 10%. For example, the arbitrary particular decrease in FEV₁ can be, for example, a decrease of about 20%. The provocator compound can be, for example, adenosine 5′-triphosphate (ATP); or an analog of ATP, such as, e.g., α,β-methylene ATP (α,βmATP); β,γ-methylene ATP ((β,γmATP); or di-adenosine pentaphosphate (Ap₅A). Analogs of ATP include other analogs having provocator activity. The administration can be by, e.g., intrapulmonary inhalation or by intravenous bolus injection.

In another aspect, the injection provides a method of therapy, the method of the therapy including: (a) performing the above-described method of diagnosis; and (b) treating the test subject for asthma or COPD. The treatment can include administering a purinergic receptor type 2 (P2R) antagonist to the test subject, e.g., a P2Y receptor antagonist and/or a P2X receptor antagonist. The treatment can involve administering to the test subject one or more corticosteroids, one or more (3-adrenosceptor agonists, or one or more anti-tussive agents. Agents useful for the method include, for example: pyridoxalphosphate-6-azophenyl-2′4′-disulphonic acid (PPADS); 5-{[3″-diphenylether (1′,2′,3′,4′-tetrahydronaphthalen-1-yl)amino]carbonyl}benzene-1,2,4-tricarboxylic acid; 2′,3′-O-(4-benzoylbenzoyl)-ATP (BzATP); tetramethylpyrazine (TMP); and 2′,3′-O-2,4,6-trinitrophenyl-ATP (TNP-ATP).

Agents useful for the treatment of OPD can also include compounds of formula (I):

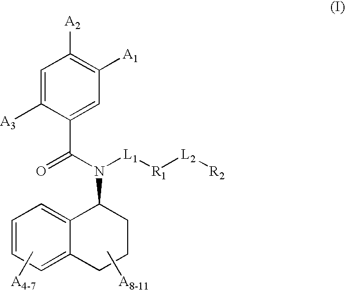

or a pharmaceutically acceptable salt thereof, wherein A₁ and A₂ are each independently selected from alkoxycarbonyl, alkylcarbonyloxy, carboxy, hydroxy, hydroxyalkyl, (NR_{A}R_{B})carbonyl, —NR_{C}S(O)₂R_{D}, —S(O)₂OH, and tetrazolyl; or A₁ and A₂ together with the carbon atoms to which they are attached form a five membered heterocycle containing a sulfur atom wherein the five membered heterocycle is optionally substituted with 1 or 2 substituents selected from mercapto and oxo; A₃ is selected from alkoxycarbonyl, alkylcarbonyloxy, carboxy, hydroxy, hydroxyalkyl, (NR_{A}R_{B})carbonyl, NR_{C}S(O)₂R_{D}, —S(O)₂OH and tetrazolyl; A₄, A₅, A₆ and A₇ are each independently selected from hydrogen, alkoxy, alkoxycarbonyl, alkenyl, alkyl, alkylcarbonyl, alkylcarbonyloxy, alkynyl, aryl, carboxy, cyano, haloalkoxy, haloalkyl, halogen, hydroxy, hydroxyalkyl, nitro, —NR_{E}R_{F}, and (NR_{E}R_{F})carbonyl; A₈, A₉, A₁₀ and A₁₁ are each independently selected from hydrogen, alkoxy, alkoxycarbonyl, alkenyl, alkyl, alkylcarbonyl, alkylcarbonyloxy, alkynyl, aryl, carboxy, haloalkoxy, haloalkyl, halogen, hydroxy, hydroxyalkyl, —NR_{E}R_{F}, (NR_{E}R_{F})carbonyl, and oxo; R_{A} and R_{B} are each independently selected from hydrogen, alkyl, and cyano; R_{R} is selected from hydrogen and alkyl; R_{D} is selected from consisting of alkoxy, alkyl, aryl, arylalkoxy, arylalkyl, haloalkoxy, and haloalkyl; R_{E} and R_{F} are each independently selected from hydrogen, alkyl, alkylcarbonyl, formyl, and hydroxyalkyl; L₁ is selected from alkenylene, alkylene, alkynylene, —(CH₂)ₘO(CH₂)ₙ—, —(CH₂)ₘS(CH₂)ₙ—, and —(CH₂)ₚC(O)(CH₂)_{q}—, wherein the left end of the group is attached to N and the right end of the group is attached to R₁; m is an integer 0-10; n is an integer 0-10; R₁ is selected from the group consisting of aryl, cycloalkenyl, cycloalkyl, and heterocycle; L₂ is absent or selected from the group consisting of a covalent bond, alkenylene, alkylene, alkynylene, —(CH₂)ₚO(CH₂)_{q}—, —(CH₂)ₚS(CH₂)_{q}—, —(CH₂)ₚC(O)(CH₂)_{q}—, —(CH₂)ₚC(OH)(CH₂)_{q}—, and —(CH₂)ₚCH═NO(CH₂)_{q}—, wherein the left end of the group is attached to R₁ and the right end of the group is attached to R₂; p is an integer 0-10; q is an integer 0-10; and R₂ is absent or selected from aryl, cycloalkenyl, cycloalkyl, and heterocycle.

Compounds of formula (I) can include, for example, 5-({(3-phenoxybenzyl)[(1S)-1,2,3,4-tetrahydro-1-napththalenyl]amino}carbonyl)-1,2,4-benzenetricarboxylic acid (A-317491) having a formula (II):

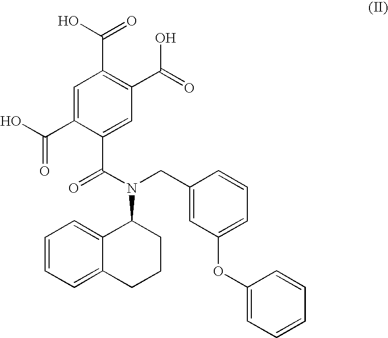

Also embodied by the invention is a method of assessing the efficacy of treatment for asthma or COPD. The method includes: (a) performing the above-described method of treatment; (b) administering a provocator compound to the test subject; (c) determining a difference in lung function, or detecting a change in at least one symptom, between before and after the administration; (d) determining whether the difference in lung function, or the change in the at least one symptom, in the test subject is closer to a mean change in lung function, or a mean difference in the at least one symptom, in control normal subjects than the difference in lung function, or in the change in the at least one symptom, in the test subject determined or detected prior to performing the treatment; and (e) classifying the treatment as effective if the difference in lung function, or the change in the at least symptom, in the test subject is closer to the mean change in lung function, or mean difference in the at least one symptom, in control normal subjects than a difference in lung function, or in a change in the at least one symptom, in the test subject determined or detected prior to performing the treatment.

Another aspect of the invention is a method of assessing the efficacy of treatment for an obstructive pulmonary disease (OPD). The method includes: (a) identifying a subject that has been treated for an OPD; (b) administering a provocator compound to the test subject; (c) determining a difference in lung function, or detecting a change in at least one symptom, between before and after the administration; (d) determining whether the difference in lung function, or the change in the at least one symptom, in the test subject is closer to the mean change in lung function, or mean difference in at least one symptom, in control normal subjects than the difference in lung function, or in the change in the at least one symptom, in the test subject determined or detected prior to the treatment for the OPD; and (e) classifying the treatment as effective if the difference in lung function, or the change in at least one symptom, in the test subject is closer to the mean change in lung function, or mean difference in at least one symptom, in control normal subjects than the difference in lung function, or in the change in the at least one symptom, in the test subject determined or detected prior to the treatment. Difference in lung function determinations, the provocator compounds, and routes of administration of provocator compounds can be as described above for the method of diagnosis. The change in at least one symptom can be, for example, a change in: Borg score; cough; chest tightness; throat tightness; sputum; or wheezing. The OPD can be, for example, asthma, COPD, or chronic cough. The subject can have had any of the treatments recited above for methods of therapy. For example, the subject can have been administered one or more compounds of formula (I), such as, for example the compound of formula (II), i.e., 5-({(3-phenoxybenzyl)[(1S)-1,2,3,4-tetrahydro-1-napththalenyl]amino}carbonyl)-1,2,4-benzenetricarboxylic acid (A-317491). The subject can, for example, have been treated with an anti-tussive agent and the at least one symptom can be cough The change in cough can be determined as a function of the amount of the provocator compound that is required to induce coughing.

Another aspect of the invention is a method of treating an OPD or cough. The method includes the steps of: (a) identifying a mammalian subject as having an OPD, having one or more symptoms associated with an OPD, or having cough;

and administering to the subject a therapeutically effective dose of a pharmaceutical composition that includes one or more compounds of formula (I). The compound can be, for example, the compound of formula (II), i.e., 5-({(3-phenoxybenzyl)[(1S)-1,2,3,4-tetrahydro-1-napththalenyl]amino}carbonyl)-1,2,4-benzenetricarboxylic acid (A-317491). The OPD can include coughing or can be, for example, COPD and asthma. The OPD can also include acute bronchitis, emphysema, chronic bronchitis, bronchiectasis, cystic fibrosis, and acute asthma, and the symptom can be, e.g., cough. The one or more of the compounds of formula (I), e.g., the compound of formula (II) (i.e., A-31749), can have the ability to inhibit vagal activation mediated by a P2R on a vagal afferent nerve terminal. The vagal afferent nerve terminal can be, for example, a C fiber terminal or an A fiber terminal. The P2R can be a P2X receptor, such as, for example, P2X₃ or P2X_{2/3}. The vagal activation can be by ATP or analogs of ATP, such as, e.g., α,βmATP or β,γmATP. The pharmaceutical composition that includes the one or more compounds can be administered by intrapulmonary inhalation or intravenous bolus injection. The composition can also be administered via any of the following routes: oral, transdermal, intrarectal, intravaginal, intranasal, intraocular, intragastrical, intratracheal, or intrapulmonary, subcutaneous, intramuscular, or intraperitoneal.

Another aspect of the invention includes a method of inhibiting activation of a P2R on pulmonary vagal sensory nerve fibers. The method includes contacting the vagal sensory nerve fiber with one or more compounds, each compound being of formula (I). The compound can be, for example, the compound of formula (II), i.e., 5-({(3-phenoxybenzyl)[(1S)-1,2,3,4-tetrahydro-1-naphthalenyl]amino}carbonyl)-1,2,4-benzenetricarboxylic acid (A-317491). Inhibiting the activation of the P2R can include inhibiting P2R-activated cation flux. The contacting of the vagal sensory nerve fiber with the composition can be in vitro or in a mammalian subject in vivo, such as, for example, a human, and can include administering the composition to the mammalian subject. The mammalian subject can have an OPD and/or cough. The composition can be administered as described above for methods of treating an OPD. The OPD can include, for example, COPD, asthma, or cough. The OPD can also include, acute bronchitis, emphysema, chronic bronchitis, bronchiectasis, cystic fibrosis, and acute asthma. The vagal sensory nerve fiber can be a C fiber or an A fiber. The P2R can be a P2X receptor, such as, for example, P2X₃ or P2X_{2/3}. The vagal activation can be in response to ATP or analogs of ATP, such as, e.g., α,βmATP, β,γmATP, or Ap5A. For the purposes of the invention, analogs of ATP have provocator activity.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., a test to distinguish asthma from COPD, will be apparent from the following description, from the drawings and from the claims.

### DESCRIPTION OF DRAWINGS

FIGS. 1A and B are scatter plots showing values of PD₂₀ obtained with individual human subjects (in the categories indicated on the x-axes) after challenge with AMP (FIG. 1A) or ATP (FIG. 1B). Horizontal solid bars indicate geometric means and dashed lines indicate the highest concentration of AMP or ATP administered to the subjects. Data from patients not responding to the highest concentration of the AMP or ATP were not included in the calculations of the geometric means.

FIGS. 2A and B are a series of line graphs showing the Borg scores obtained from individual subjects (in the categories indicated) before (“B/L”; baseline), immediately after challenge with a PD₂₀ concentration of AMP (FIG. 2A) or ATP (FIG. 2B) (“PD₂₀”), and 30 minutes after the challenge.

FIGS. 3A and B are a pair of bar graphs showing mean Borg scores of patients with asthma (“Asthma”) or COPD (“COPD”) after challenge with AMP (FIG. 3A) or ATP (FIG. 3B).

FIGS. 4A and B are a pair of bar graphs showing mean changes in Borg score (“ΔBorg”) of subjects (in the categories indicated on the x-axes) after challenge with AMP (FIG. 4A) or ATP (FIG. 4B).

FIGS. 5A and B are a pair of bar graphs showing the percentage of subjects (in the categories listed in the bar fill key) having the symptoms listed on the x-axes after challenge with AMP (FIG. 5A) or ATP (FIG. 5B).

FIGS. 6A and B are a pair of scatter plots showing the relationship between change in FEV₁ (“% fall in FEV₁”) and Borg score in subjects administered a PD₂₀ dose of AMP (FIG. 6A) or ATP (FIG. 6B) (“Borg score at PD₂₀”).

FIG. 7 is a recorder trace showing action potentials in a dog pulmonary rapidly adapting receptor (RAR)-containing afferent nerve fiber before and after exposure of the dog to ATP. The time at which the dog was exposed to ATP is indicated by the term “ATP” over an inverted triangle. Action potential volleys due to individual respiratory cycles are indicated by upwardly pointing arrows.

FIG. 8 is a recorder trace showing action potentials in a dog RAR-containing afferent nerve fiber before and after exposure of the dog to capsaicin. The time at which the dog was exposed to capsaicin is indicated by the term “Capsaicin” over an inverted triangle. Action potential volleys due to individual respiratory cycles are indicated by upwardly pointing arrows.

FIG. 9 is a series of recorder traces showing action potentials in a dog vagal RAR-containing nerve fiber and a vagal C fiber before and after exposure of the dog to ATP, capsaicin, β,γmATP, or α,βmATP. The time point at which the dog was exposed to the various provocator compounds is indicated by an inverted triangle. Action potential volleys due to individual respiratory cycles are indicated by downwardly pointing arrows. Segments of the traces corresponding to RAR-associated responses and C fiber responses are indicated by brackets and “Aδ” and “C”, respectively.

FIG. 10. is a graph showing the number of action potentials in A fiber (left graph) and C fiber (right graph) terminals measured in a guinea pig perfused nerve-lung preparation in response to treatment with α,βmATP alone (control) or in combination with 1 uM or 10 uM of the selective P2X₃/P2X₂₁₃ receptor antagonist A-317491. The action potentials were quantified as discharge/sec and are depicted as mean±standard deviation (SD).

### DETAILED DESCRIPTION

There are three major sensory pathways carrying afferent neural traffic from the lungs to the brain: C fibers, slowly adapting receptor (SAR)-containing fibers and rapidly adapting receptor (RAR)-containing fibers. C fibers are non-myelinated, slowly conducting fibers that are quiescent unless stimulated. Their terminals contain bimodal receptors that respond to chemical and mechanical stimuli. SAR and RAR-containing fibers are rapidly conducting myelinated fibers that are activated during each respiratory cycle. Multiple endogenous and exogenous compounds stimulate C fibers and RAR-containing fibers; capsaicin, the active ingredient in red pepper, stimulates C fibers but not RAR-containing fibers as the latter lack the capsaicin receptor (the valinoid receptor, VR-1).

Adenosine 5′-triphosphate (ATP) is a purine nucleotide found in every living cell where it plays a critical role in cellular metabolism and energetics. ATP is released from cells under physiologic and pathophysiologic conditions; extracellular ATP acts as a local physiologic regulator as well as an endogenous mediator that plays a mechanistic role in the pathophysiology of obstructive airway diseases [Pelleg et al. (2002) Am. J. Ther. 9:454-464]. ATP exerts potent effects on dendritic cells, eosinophils and mast cells. For example, it enhances IgE-mediated release of histamine and other mediators from human lung mast cells [Schulman et al. (1999) Am. J. Respir. Cell. Mol. Biol. 20:530-537]. Extracellular ATP also exacerbates neurogenic bronchoconstriction and inflammation by stimulating vagal sensory (afferent) nerve terminals in the lungs and stimulating the release of neuropeptides [Pelleg et al. (2002), supra; Schulman et al. (1999), supra; Katchanov (1998) Drug Dev. Res. 45:342-349]. It has been shown that patients with asthma exhibit a more intense response (i.e., bronchoconstriction) to inhaled ATP than normal individuals and, in both groups of subjects, ATP was more potent than methacholine and histamine [Pellegrino et al. (1996) J. Appl. Physiol. 81: 342-349].

Adenosine is a purine nucleoside that is a product of the enzymatic degradation of ATP. Aerosolised adenosine causes bronchoconstriction in asthmatic but not healthy subjects [Cushley et al. (1983) Br. J. Clin. Pharmacol. 15:161-165]. Since the dose-response curves for adenosine and adenosine 5′-monophosphate (AMP) with respect to their ability to induce bronchoconstriction in asthmatic patients are identical [Mann et al. (1986) J. App. Physiol. 61:1667-1676], it has been concluded that they act by the same pathway. The action of AMP is likely mediated by adenosine produced by AMP degradation by ecto-enzymes. In addition, since AMP is much more soluble than adenosine, AMP has been used instead of adenosine in the clinical setting. The effects of AMP and adenosine on airway smooth muscle cells are mediated by mast cells and inflammatory mediators released from these cells.

Extracellular ATP affects many cell types in different tissues and organs by activating cell surface receptors known as P2 purinergic receptors (P2R), and in particular the P2X class of P2R. P2R are distinct from the P1 purinergic receptors (P1R), which are adenosine receptors. P2R are divided into two families: P2X, ligand-binding, dimeric, trans-cell membrane cationic channels, and P2Y, seven trans-cell membrane domain G protein-coupled receptors. Eight P2Y (P2Y₁, P2Y₂, P2Y₄, P2Y₆, P2Y₁₁, P2Y₁₂, P2Y₁₃, and P2Y₁₄), seven homodimeric P2X receptor subtypes (P2X₁₋₇), and five P2X heterodimeric receptors (X_{1/2}, X_{2/3}, X_{2/6}, X_{1/5}, and X_{1/6}) have been identified and cloned. In general, the stimulation of the P2Y receptors activates an intracellular signal transduction pathway culminating in the increase in the level of intracellular calcium (Ca²⁺) ions.

Aerosolised ATP, but not AMP/adenosine, causes bronchoconstriction, in healthy subjects. In addition, ATP, but not adenosine, activates vagal sensory nerve terminals in the lungs (C fibers as well as RAR-containing fibers). ATP stimulates this activity via a subclass of P2X receptors [Pelleg et al (1996) J. Physiol. 490(1):265-275; U.S. Pat. No. 5,874,420; Example 5 and 6 below].

The studies described in the Examples below provide further evidence of the qualitative difference between the mammalian pulmonary response to ATP and that to AMP and thus also of such a difference between the pulmonary response to ATP and that to adenosine. These studies also provide a rationale for using compounds that selectively inhibit the activation of P2R localized on vagal sensory nerve terminals, particularly in response to ATP. Importantly, these studies provide the basis for methods for establishing whether a mammalian subject has asthma or COPD, for treating OPD such as asthma and COPD, for treating cough, and for assessing the efficacy of a treatment for an OPD. The invention also includes a method for inhibiting activation of a P2R on a pulmonary vagal sensory nerve fiber. These methods are described below.

### Methods of Diagnosis, Treatment, and Assessing the Efficacy of Treatment

Included in the invention are methods: (a) to distinguish asthma from COPD; (b) to treat subjects with an OPD; and (c) to assess the efficacy of a particular treatment regimen. Methods of treatment and methods to assess the efficacy of a particular treatment can be given without, or with, first performing one or more of the methods to distinguish asthma from COPD. In addition, the methods to assess the efficacy of treatment can be used after performing one or more of the treatment methods described below or any other method of treatment known in the art.

Methods of Diagnosis

In a method of diagnosis, a provocator compound is administered to a subject known to have either asthma or COPD and the effect of the compound on lung function in the subject is determined. It is understood that the term “determining lung function” in this context preferably involves actively performing a test (e.g., spirometry or plethysmography) that objectively assesses lung function. Less preferable tests include detecting pulmonary symptoms such as coughing, sputum, chest tightness, throat irritation, wheezing, or Borg score. So determining a “difference in lung function” means determining a difference in lung function as measured by any of the tests described above.

Useful provocator compounds include ATP and related compounds (analogs) such as: α,β-methylene-ATP (α,βmATP); β,γ-methylene-ATP (β,γmATP); 2-methylthio-ATP; and di-adenosine pentaphosphate (Ap₅A). The provocator compounds can be used singly or in combinations of, for example, two, three, four, or five. As used herein, a “provocator compound” is a compound that when administered to a mammalian subject (e.g., a human) results in significantly decreased lung function. Provocator compounds can act, for example, by stimulating P2R (e.g., P2X₂₃ receptors) on the terminals of vagal nerve fibers in the lung. “Significantly decreased lung function” means a decrease in function of at least 5% (e.g., at least 10%, at least 20%, at least 30%, at least 40%, at least 50%, at least 60%, at least 70%, at least 80%, at least 90%, or at least 100%). The decrease in lung function can be evidenced by, for example, bronchial constriction, coughing, wheezing, or any of the symptoms recited herein.

Subjects can be of any mammalian species that is susceptible to an OPD such as asthma, COPD, or chronic cough, e.g., humans, non-human primates (e.g., monkeys, gorillas, and baboons), horses, bovine animals (e.g., cows, bulls, and oxen), sheep, goats, pigs, dogs, cats, rabbits, guinea pigs, hamsters, gerbils, rats, and mice. Subjects are preferably human patients.

The route of administration of a provocator compound can be any route that results in contact of the compound with the compound's site of action (i.e., the lungs) in the body of a subject. Appropriate routes of administration include, but are not limited to, intrapulmonary (e.g., inhalative), oral, topical, hypodermal, intradermal, subcutaneous, transcutaneous, intravenous (e.g., intravenous bolus), intramuscular, and intraparenteral methods of administration. The administration is preferably intrapulmonary, e.g., as an aerosolized intrapulmonary puff.

It is contemplated that the dosage of a provocator compound will be in the range of from about 0.1 ug to about 100 mg per kg of body weight, preferably from about 10 ug to about 20 mg per kg. Pharmaceutical compositions containing the provocator compounds may be administered in a single dosage, plural dosages, or by sustained release. The provocator compounds can be administered as a bolus. Persons of ordinary skill will be able to determine dosage forms and amounts with routine experimentation based upon the teachings herein and the personal knowledge of such persons.

Where the route of administration is intrapulmonary, the composition containing one or more provocator compounds can be administered using any of a variety of inhalers known in the art, e.g., a portable propellant-based inhaler. Alternatively, it can be administered in a nebulized composition by, for example, a nebulizer connected to a compressor.

In the provocator compound compositions, the compounds can be dispersed in a solvent, e.g., in the form of a solution or a suspension. They can be dispersed in an appropriate physiological solution, e.g., physiological saline. The compositions can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, glucose, mannitol, sorbitol, glycerol, or a glycol such as propylene glycol or polyethylene glycol. Solutions or suspensions can be encapsulated in liposomes or biodegradable microspheres. Suitable preservatives include benzalkonium chloride, methyl- or propyl-paraben, and chlorobutanol. Pharmaceutical formulations are known in the art, see, for example, Gennaro Alphonso, ed., Remington's Pharmaceutical Sciences, 18^{th} Ed., (1990) Mack Publishing Company, Easton, Pa.

In the diagnostic method of the invention, the effect of the compound on lung function can be determined by any of a variety of methods known in the art. Lung function is determined before and after, and optionally during, administration of a provocator compound. It can be determined immediately after or a significant time after administration of a provocator compound. Thus lung function can be determined, as appropriate, from one or two seconds to several months (e.g., 10 seconds, 20 seconds, 30 seconds, 45 seconds, one minute, two minutes, five minutes, 10 minutes, 20 minutes, 30 minutes, 45 minutes, one hour, two hours, three hours, five hours, eight hours, 10 hours, 12 hours, 15 hours, 18 hours, one day, two days, three days, four days, five days, six days, seven days, ten days, two weeks, three weeks, one month, two months, three months, four months, five months, or six months) after administration of a provocator compound.

Determination of lung function can be quantitative, semi-quantitative, or qualitative. Thus it can, for example, be measured as a discrete value. Alternatively, it can be assessed and expressed using any of a variety of semi-quantitative/qualitative systems known in the art. Thus, lung function can be expressed as, for example, (a) one or more of “excellent”, “good”, “satisfactory”, “unsatisfactory”, and/or “poor”; (b) one or more of “very high”, “high”, “average”, “low”, and/or “very low”; or (c) one or more of “++++”, “+++”, “++”, “+”, “+/−”, “−”.

The change in level of lung function in the test subject due to the action of the provocator compound is then compared to mean changes in levels of lung function obtained from panels of control patients having either asthma or COPD. If the change in level of lung function in the test subject is closer to the mean change in level of lung function in control asthmatic patients than that in control COPD patients, it is likely that the test subject has asthma. On the other hand, if the change in level of lung function in the test subject is closer to the mean change in level of lung function in control COPD patients than that in control asthma patients, it is likely that the test subject has COPD.

Thus, for example, the effect of a provocator compound on the level of forced expired volume in one second (FEV₁; the volume expired in the first second of maximal expiration after a maximal inspiration) can be tested by any means known in the art. For example, the change in lung function can be expressed as the concentration (or amount) of the provocator compound required to cause an arbitrarily defined decrease in FEV₁ (e.g., see Example 2). The arbitrarily defined decrease in FEV₁ can be, for example, a decrease of about: 5%; 10%; 15%; 20%; 25%; 30%; 35%; 40%; 45%; or 50%. As used in this context, “about” means that the arbitrarily defined decrease in FEV₁ can vary by 1-4 percentage points from the stated percentage. Thus, for example, a decrease of about 20% can be a decrease of from 16%-24%.

Alternatively, for example, the effect of a fixed dose of the provocator compound on the FEV₁ level can be measured. In addition, the FEV₁ value can expressed as a percentage of the FVC (forced vital capacity; maximum volume of air that can be exhaled during a forced maneuver).

Other parameters indicative of lung function can be employed, e.g., specific airway conductance (sGaw), Borg score, functional residual capacity (FRC), forced expiratory flow (FEF), and peak expiratory flow rate (PEFR), using either the first approach (measuring the amount of a provocator compound necessary to cause an arbitrary change in the level of the parameter of interest) or the second approach (determining the effect of a fixed dose of a provocator compound on the level of the parameter of interest) described above. Those skilled in the art are familiar with methods of determining these parameters. Changes are decreases or increases, depending on the parameter being determined. The arbitrarily defined change in a parameter can be, for example, a change of about: 5%; 10%; 15%; 20%; 25%; 30%; 35%; 40%; 45%; or 50%.

Methods of Treatment

Treatment methods can be any methods of treating cough or an OPD, e.g., asthma, COPD, or chronic cough, in any of the mammalian subjects listed in Methods of Diagnosis. Useful therapeutic agents include, for example, oral and/or inhaled corticosteroids, beta adrenoceptor agonists, anti-cholinergic agents, leukotriene antagonists, antibodies (e.g., polyclonal or monoclonal antibodies such as a humanized monoclonal antibody) specific for immunoglobulin E (IgE), tyrosine kinase inhibitors, theophylline, or anti-tussive agents [Tamul et al. (2004) Crit. Care. Med. 32 (4 Suppl):S137-S145; Frew (2004) Clin. Allergy Immunol. 18:561-566; Allen-Ramey (2004) Ann. Epidemiol. 14:161-167; Wong et al. (2004) Biochim. Biophys. Acta. 1697:53-69; Hansel et al. (2004) Drugs Today (Barc) 40:55-69; Vignola (2003) Drugs. 63 (Suppl 2):35-51; Creticoa Drugs 63 (Suppl 2):1-20]. They also include, for example, those in which a P2-purinoreceptor (P2R) antagonist is administered to a subject with cough or with an OPD, e.g., asthma, COPD, or chronic cough.

As used herein, the term “P2R antagonist” includes agents that: (a) inhibit activation by a P2R agonist of cells expressing a P2R; or (b) inhibit the activity of a cell expressing a P2R. Such P2R antagonists can act by completely or substantially inhibiting binding of an agonist to the P2R by binding to the binding site on the P2R of the relevant agonist or they can act allosterically by binding at a site other than binding site on the P2R of the agonist and inducing a conformational change in the P2R such that binding of an agonist to the P2R is substantially, if not completely, inhibited. Alternatively, a P2R antagonist can inhibit an activity of a cell expressing a P2R by binding to the P2R, either at an agonist-binding site or at a separate site, and delivering an inhibitory signal to the cell. Alternatively, P2R antagonists can act at sites downstream from the P2R by interfering with one or more steps of the relevant signal transduction initiated by the P2R.

P2R antagonists useful in the invention include P2X receptor antagonists and P2Y receptor antagonists. Examples of P2X inhibitors include, for example: pyridoxalphosphate-6-azophenyl-2′4′-disulphonic acid (PPADS); 5-{[3″-diphenylether (1′,2′,3′,4′-tetrahydronaphthalen-1-yl)amino]carbonyl}benzene-1,2,4-tricarboxylic acid; 2′,3′-O-(4-benzoylbenzoyl)-ATP (BzATP); tetramethylpyrazine (TMP); 2′,3′-O-2,4,6-trinitrophenyl-ATP (TNP-ATP). Importantly it was shown that PPADS reduced the number of vagal action potentials elicited by administration of ATP to a dog (see U.S. Pat. No. 5,874,420, which is incorporated herein by reference in its entirety).

P2Y receptor antagonists can also be useful for treating asthma, COPD, and/or chronic cough. For example, N⁶-methyl 2′-deoxyadenosine 3′,5′-bisphosphate; β,γ-imido-ATP; and diadenosine-n (4-6)-phosphate are antagonists of P2Y₁. ATP is an antagonist of P2Y₄ and the compounds AR-C69931 MX (Astra-Zeneca) and AR-66096 (Astra-Zeneca) are potent antagonists of the P2Y₁₂ receptor [Shaver S R (2001) Curr. Opin. Drug Disc. Dev. 4:665-70]. The compound 2,2′-pyridylisatogen tosylate (PIT) is also an antagonist and allosteric modifier of P2Y receptors [Spedding (2000) J. Auton. Nerv. Sys. 81:225-7]. The above-mentioned ability of ATP to enhance histamine release by IgE-activated lung mast cells is mediated by P2Y receptors and it is likely thus P2Y receptor antagonists would be particularly efficacious in the treatment of asthma.

Also of interest for the treatment of an OPD or cough are certain non-nucleotide antagonists of P2R. For example, a family, of non-nucleotide antagonists that have high affinity and selectivity for blocking P2X₃ and P2X_{2/3} receptors and dose-dependently reduce nociception in neuropathic and inflammatory animal pain models (see, e.g., Jarvis et al. (2002) PNAS. 99(26):17179-17184 and U.S. Pat. No. 6,831,193 B2, which are both incorporated herein by reference in their entirety) are of particular interest. One of these compounds, A-317491, was determined to be highly effective at inhibiting vagal sensory nerve responses in C and A fibers activated by α,βmATP (see Example 6 below). Of particular interest are P2X₃ and P2X_{2/3} receptor antagonists. Examples of such compounds are those described in U.S. Pat. No. 6,831,193, which are of formula (I):

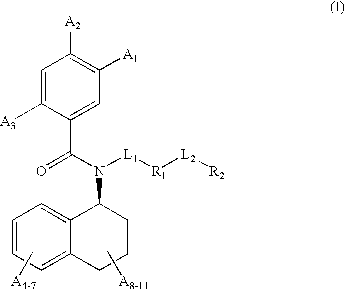

or a pharmaceutically acceptable salt thereof, wherein A₁ and A₂ are each independently selected from alkoxycarbonyl, alkylcarbonyloxy, carboxy, hydroxy, hydroxyalkyl, (NR_{A}R_{B})carbonyl, —NR_{C}S(O)₂R_{D}, —S(O)₂OH, and tetrazolyl; or A₁ and A₂ together with the carbon atoms to which they are attached form a five membered heterocycle containing a sulfur atom wherein the five membered heterocycle is optionally substituted with 1 or 2 substituents selected from mercapto and oxo; A₃ is selected from alkoxycarbonyl, alkylcarbonyloxy, carboxy, hydroxy, hydroxyalkyl, (NR_{A}R_{B})carbonyl, NR_{C}S(O)₂R_{D}, —S(O)₂OH, and tetrazolyl; A₄, A₅, A₆ and A₇ are each independently selected from hydrogen, alkoxy, alkoxycarbonyl, alkenyl, alkyl, alkylcarbonyl, alkylcarbonyloxy, alkynyl, aryl, carboxy, cyano, haloalkoxy, haloalkyl, halogen, hydroxy, hydroxyalkyl, nitro, —NR_{E}R_{F}, and (NR_{E}R_{F})carbonyl; A₈, A₉, A₁₀ and A₁₁ are each independently selected from hydrogen, alkoxy, alkoxycarbonyl, alkenyl, alkyl, alkylcarbonyl, alkylcarbonyloxy, alkynyl, aryl, carboxy, haloalkoxy, haloalkyl, halogen, hydroxy, hydroxyalkyl, —NR_{E}R_{F}, (NR_{E}R_{F})carbonyl, and oxo; R_{A} and R_{B} are each independently selected from hydrogen, alkyl, and cyano; R_{C} is selected from hydrogen and alkyl; R_{D} is selected from consisting of alkoxy, alkyl, aryl, arylalkoxy, arylalkyl, haloalkoxy, and haloalkyl; R_{E} and R_{F} are each independently selected from hydrogen, alkyl, alkylcarbonyl, formyl, and hydroxyalkyl; L₁ is selected from alkenylene, alkylene, alkynylene, —(CH₂)ₘO(CH₂)ₙ—, —(CH₂)ₘS(CH₂)ₙ—, and —(CH₂)ₚC(O)(CH₂)_{q}—, wherein the left end of the group is attached to N and the right end of the group is attached to R₁; m is an integer 0-10; n is an integer 0-10; R₁ is selected from the group consisting of aryl, cycloalkenyl, cycloalkyl, and heterocycle; L₂ is absent or selected from the group consisting of a covalent bond, alkenylene, alkylene, alkynylene, —(CH₂)ₚO(CH₂)_{q}—, —(CH₂)ₚS(CH₂)_{q}—, —(CH₂)ₚC(O)(CH₂)_{q}—, —(CH₂)ₚC(OH)(CH₂)_{q}—, and —(CH₂)ₚCH═NO(CH₂)_{q}—, wherein the left end of the group is attached to R₁ and the right end of the group is attached to R₂; p is an integer 0-10; q is an integer 0-10; and R₂ is absent or selected from aryl, cycloalkenyl, cycloalkyl, and heterocycle.

The chemical nomenclature use above (and throughout the specification and the appended claims) in regard to compounds of formula (I) is that used in U.S. Pat. No. 6,831,193 B2 (incorporated herein in its entirety), e.g., at column 18, line 57 to column 24, line 22.

Compounds of formula (I) can include, for example, a compound of formula (II), i.e., 5-({(3-phenoxybenzyl)[(1S)-1,2,3,4-tetrahydro-1-napththalenyl]amino}carbonyl)-1,2,4-benzenetricarboxylic acid (A-317491):

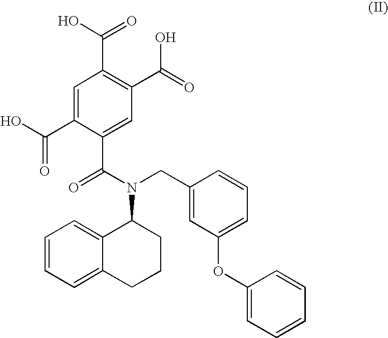

Additional compounds of formula (I) useful for these methods of the invention are listed in U.S. Pat. No. 6,831,193 B2, the disclosure of which is incorporated herein by reference in its entirety.

Therapeutic agents can be administered singly or in combination, e.g., in combinations of two, three, four, five, six, seven, eight, nine, ten, 11, 12, 15, 18, 20, or 25. Subjects, routes of administration, and formulations of the agents compositions (e.g., pharmaceutical compositions) for use in methods of treatment are the same as those for the diagnostic methods (see above).

For example, pharmaceutical compositions can be administered intrapulmonarally (e.g., inhalatively) orally, rectally, parenterally, intravaginally, intraperitoneally, topically (as powders, ointments, or drops), bucally or as an oral or nasal spray. Parenteral administrations can include intravenous, intramuscular, intraperitoneal, intrasternal, subcutaneous, and intraarticular injections and infusions. Oral administration of the composition includes solid and liquid forms. Solid administration of the composition includes, e.g., capsules, tablets, pills, powder, and granules, whereas liquid administration can include emulsions, solutions, suspensions, syrups, and elixirs.

Pharmaceutical compositions containing the agents can be administered in the form of a powder, spray, ointment, and inhalant. The one or more agents of the pharmaceutical composition can be mixed under sterile conditions with a pharmaceutically acceptable carrier and any needed preservatives, buffers, or propellants. The pharmaceutical composition also can contain one or more pharmaceutical excipients. In such compositions, the compounds can be dispersed in a solvent, e.g., in the form of a solution or a suspension. Pharmaceutical excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), ethylene diamine tetraacetic acid (EDTA), sodium chloride, liposomes, glucose, mannitol, sorbitol, glycerol, or a glycol such as propylene glycol or polyethylene glycol.

A “therapeutically effective dose” of the above described pharmaceutical composition can be determined by varying the dose administered so as to obtain an amount of the active compound(s) which is effective to achieve the desired therapeutic response for a particular subject. A desired therapeutic effect can be, for example, decreasing the severity of, or completely eradicating, the OPD, the symptoms associated with the OPD, or cough in the subject who has undergone treatment with one or more of the agents described in the present invention. The selected dosage will depend upon a variety of factors, including the activity of the particular compound, the route of administration, the severity of the OPD or cough condition being treated, the condition and the prior medical history of the subject undergoing treatment, the age, body weight, general health, sex, and diet of the subject being treated, the duration of the treatment, drugs used in combination or coincidental with the specific compound employed, and like factors were known in the medical and veterinary arts. It is contemplated that the dosage of a therapeutic agent used in the method of treatment will be in the range of from about 0.1 ug to about 100 mg per kg of body weight, preferably from about 10 ug to about 20 mg per kg per day. It may be necessary in some circumstances to deliver a daily dose in, for example, two, three, four, five, six, seven, eight, nine, ten, 11, 12, 13, 14, 16, 17, 18, 19, 20, 21, 22, 23, 24, or even more separate administrations. However, it will not always be necessary that the subject receive an agent daily. It may be required to administer the agent only once every two days, once every three days, once every four days, once every five days, once very six days, once a week, once every 10 days, once every two weeks, once every three weeks, or once a month, once every two months, or once every three months, or even once every six months. Pharmaceutical compositions may be administered in a single dosage, divided dosages or by sustained release. Persons of ordinary skill will be able to determine dosage forms, amounts, and frequency of administration by routine experimentation.

As used herein, an agent that is “therapeutic” is an agent that causes a complete abolishment of the symptoms of a disease or a decrease in the severity of the symptoms of the disease. “Prevention” means that symptoms of the disease (e.g., COPD, asthma, or cough) are essentially absent. As used herein, “prophylaxis” means complete prevention of the symptoms of a disease, a delay in onset of the symptoms of a disease, or a lessening in the severity of subsequently developed disease symptoms.

Therapeutic agents useful for all the treatment methods described in this document can be used in the manufacture of medicaments for treatment of cough, an OPD (e.g., asthma, COPD, chronic cough), or any other pathologic condition disclosed herein.

### Methods of Inhibiting P2R Activation

The invention also includes a method of inhibiting activation of a P2R on a pulmonary vagal sensory nerve fiber. The method includes contacting the vagal sensory nerve fiber (by, for example, contacting the P2R) with one or more of the above described P2R antagonists, e.g., the compounds of formula (I). The contacting can be in a mammalian subject, such as, for example, any of those described above for Methods of Diagnosis. For such in vivo methods, compositions containing P2R antagonists, formulations of such compositions, and routes and frequency of administration of the compositions are the same as those described above for Methods of Treatment. The mammalian subject whose vagal sensory nerve fiber(s) is contacted with the above described compounds can have an OPD, a symptom associated with an OPD, or cough. The OPD can be, for example, COPD, asthma, acute bronchitis, emphysema, chronic bronchitis, bronchiectasis, cystic fibrosis, and acute asthma; and symptoms include, for example, coughing, shortness of breath, and wheezing. The P2R can be any of those recited herein (e.g., P2X_{2/3} receptors).

The method of inhibiting activation of a P2R on a pulmonary vagal sensory nerve fiber can also be in vitro. In vitro applications of the methods can be useful in basic scientific studies of nerve activity, mechanisms of initiation of vagal nerve action potential and neural afferent traffic, and mechanisms of P2R activation and/or signaling. In vitro methods can also be “positive controls” in in vitro screens or tests of other compounds for their ability to inhibit activation of a P2R on a pulmonary vagal sensory fiber. In the in vitro methods of the invention, one or more of the inhibitory agents can be applied directly to an isolated nerve fiber. Thus, for example, one or more of the agents can be injected or infused via the trachea or the pulmonary artery in, for example, an in vitro perfused nerve-lung preparation obtained from an appropriate mammalian subject, such as the preparation described in Example 6.

### Methods of Assessing the Efficacy of a Treatment

Methods of assessing the efficacy of a treatment for an OPD will by definition follow treatment for the relevant OPD. The subjects can be any of those listed herein and the OPD can be any OPD, e.g., asthma, COPD, or chronic cough. Such treatments can be any of those described above. The assessment of efficacy can be carried out within one minute to one year of giving the treatment, e.g., within one minute to one hour, one hour to 24 hours, one day to one week, one week to one month, one month to six months, or six months to one year of the treatment. The assessment can be made once or a plurality of times, each time being separated by any of the time intervals listed immediately above.

One or more of the above-described provocator compounds is administered to the test subject as described above for the method of diagnosis. A determination of lung function (as described for the method of diagnosis) and/or a determination of symptoms (e.g., Borg score, cough, sputum, wheezing, chest tightness, or throat irritation) or changes in any of these symptoms due to the action of the provocator compound is then made. The test subject level is then compared to a mean level obtained from a plurality of appropriate control normal subjects. As used herein “control normal subjects” are subjects of the same species as the test subject and that do not have an OPD. Appropriate control normal subjects can be any subjects within this definition, regardless of any other characteristics. Alternatively, control normal subjects can be further broken down into subgroups according to other characteristics such as, for example, age, sex, and smoking status. Thus, for example, where the test subject is a smoker, the control normal subjects can be smokers without an OPD. Similarly, where the test subject is a non-smoker, the control normal subjects can be non-smokers without an OPD. Control normal subjects can be even further broken down, as appropriate, into, for example, heavy, light, long-term, and/or short-term smokers.

A level obtained from a test subject that deviates significantly from a mean control normal level in the direction of a mean value obtained from subjects with the relevant OPD is an indication that the relevant treatment did not eliminate the symptoms of the OPD. Such a finding can be indicative of a poor prognosis for the subject and/or the need for further treatment of the subject. On the other hand, a level obtained from the test subject that is closer to, or insignificantly different from, a mean normal control level than a level that was obtained from the test subject prior to the treatment would be and indication that the treatment was partially effective or completely effective, respectively.

Alternatively or in addition, a control level (of lung function or a symptom such as cough) to which an “after treatment” level is compared can be a level determined in the relevant subject before the treatment of interest. Thus, for example, a subject that has been treated with an agent to suppress cough (i.e, an anti-tussive agent) can be tested before and after treatment for the relative ability of a provocator to induce cough. Such a test can be, for example, one analogous to that described in Example 2. Increasing concentrations of the provocator can be administered to the subject until cough is induced. If a greater concentration of the provocator is required to induce cough after the treatment than before the treatment, it could be concluded that the treatment was effective. On the other hand, if the same or even a lower concentration of the provocator was required to induce cough after treatment than before treatment, it could be concluded that the treatment was not effective and possibly deleterious.

Determinations of lung function and assessment of symptoms can quantitative, semi-quantitative, or qualitative (see Methods of Diagnosis). Moreover the assessment of a symptom can be in terms of “presence” or “absence” of the relevant symptom.

The following examples serve to illustrate, not limit, the invention.

### EXAMPLES

### Example 1

### Materials and Methods

### Patients in Clinical Study Described in Examples 2-4

Healthy non-smokers (age 41±3 yrs, n=10, six males), patients with intermittent asthma (age 39±3 yrs, n=10, seven males), healthy current smokers (age 40±4 yrs, n=7, five males), smokers at risk of developing COPD (age 50±4 yrs, n=7, four males) and patients with mild to moderate COPD (age 58±4 yrs, n=7, four males, two mild and five moderate were included in this study (Table 1).

TABLE 1
Characteristics of Patients in Study Described in Examples 2-4

| | Non- | | Healthy | Smokers | |
|---|---|---|---|---|---|
| | smokers | Asthma | smokers | at risk | COPD |
| | (n = 10) | (n = 10) | (n = 7) | (n = 7) | (n = 7) |
| Age, y | 41 ± 3 | 39 ± 3 | 40 ± 4 | 50 ± 4 | 58 ± 4 |
| Male/Female | 6/4 | 7/3 | 5/2 | 4/3 | 4/3 |
| Non-smokers | 10 | 8 | — | — | — |
| Ex-smokers | — | 2 | — | — | 2 |
| Current smokers | — | — | 7 | 7 | 5 |
| Pack-years | — | 2 ± 1 | 19 ± 3 | 34 ± 2 | 46 ± 9 |
| FEV₁, % pred | 103 ± 4 | 91 ± 4 | 104 ± 3 | 104 ± 3 | 76 ± 4 |
| FEV₁/FVC, % | 85 ± 1 | 78 ± 2 | 82 ± 1 | 81 ± 1 | 68 ± 3 |
| Skin prick test, | | | | | |
| Positive | 4 | 9 | 4 | 1 | 1 |
| Negative | 6 | 1 | 3 | 6 | 6 |

Values are expressed as number or mean ± SEM (standard error of the mean).

Healthy non-smokers and smokers had no history of any respiratory symptoms or respiratory infection and normal lung function without reversibility. Smoking subjects had a history of >10 cigarette pack-years (i.e., more than one pack per day for 10 years). The diagnoses of COPD and smokers at risk were based on the GOLD guideline [Pauwels et al. (2001) Am. J. Respir. Crit. Care Med. 163:1256-1276] and the GINA guideline [Liard et al. (2000) Eur. J. Respir. 16:615-620] was used to define asthmatic patients. Patients with asthma and COPD were clinically stable with no changes in symptoms and medication, no respiratory tract infection, and no use of steroids in the preceding four weeks.

The study was approved by the ethics committee of the Royal Brompton Hospital and Harefield NHS Trust, London, England, and all subjects gave written informed consent.

### Design of Clinical Study Described in Examples 2-4

The clinical study described in Examples 2-4 was randomized, double blind, crossover, and controlled. Each subject attended the laboratory on three occasions. Procedures on the initial screening visit included obtaining information about the study and a number of investigations in the following order: medical history, lung function, reversibility and skin prick testing. At visit 2 and 3, separated by 2-7 days, the subjects were subjected to either an ATP or AMP challenge in a randomized fashion such that, by the end of the study, each patient had been tested with both compounds. Before, immediately after, and 30 minutes after the challenge, lung function and Borg score for dyspnoea were determined, and symptoms other than dyspnoea were recorded.

### Skin Prick Testing

In all the subjects of the clinical study described in Examples 2-4, skin sensitivity to four common aeroallergens (house dust mite, grass pollen, cat hair and Aspergillus fumigatus, with negative and positive controls; Soluprick™, ALK-Abelló A/S, Hørsholm, Denmark) were performed. Wheal size was determined 15 minutes later and a positive reaction was recorded in the presence of at least one wheal 3 mm larger than negative control.

Pre-ATP/AMP Challenge Test Assessment of Lung Function

In the clinical study described in Examples 2-4, spirometric tests were performed using a dry spirometer (Vitalograph Ltd., Buckingham, England) and the best value of the three expiratory manoeuvers was expressed as liters and percentage of the predicted. Subjects were then given salbutamol (200 ug) by metered dose inhaler through a spacer, and lung function determination was repeated 15 minutes later, an increase in FEV₁ that was greater than 200 ml and 12% was considered reversible [Pauwels et al., supra].

### Borg Score

The modified Borg scale used to quantitate dyspnoea in the clinical study described in Examples 2-4 was a category scale in which words describing degrees of breathlessness were anchored to numbers between 0 and 10. The subjects were asked to select a number whose corresponding words most appropriately described their perception of breathlessness. The change in dyspnoea was expressed as ΔBorg, which was the difference in Borg score before and after the challenge [Rutgers et al. (2000) Eur. Respir. J. 16:486-490; Burdon et al. (1982) Am. Rev. Respir. Dis. 126:825-828]. Terms and associated numbers used in assessing Borg score were as follows: 0, “nothing at all”; 0.5, “very, very slight”; 1, “very slight”; 2, “slight”; 3, “moderate”; 4, “somewhat severe”; 5, “severe”; 7, “very severe”; 9, “very, very severe (almost maximal)”; and 10, “maximal” [Burdon et al. (1982)].

### Inhalation Challenge Tests

For the tests described in Examples 2-4, ATP and AMP (Sigma, Gillingham, Dorset, England) were dissolved freshly in normal saline solution to produce a range of doubling concentrations from 0.227-929 umol/ml for ATP and from 0.138-1152 umol/ml for AMP and immediately used for bronchial challenge. The solutions were administered using a breath-activated dosimeter (Mefar, Bovezzo, Italy) with an output of 10 ul per inhalation [Prieto et al. (2003) Chest 123:993-997]. The subjects, while wearing a nose clip, inhaled (via a mouthpiece) five breaths of normal saline, followed by sequential doubling concentrations of either ATP or AMP from functional residual capacity to total lung capacity. FEV₁ was measured from two minutes after the fifth inhalation of normal saline until there was a fall in FEV₁ of ≧20% of its value recorded after saline inhalation or until maximal concentration of either ATP or AMP was inhaled. The provocative dose causing a 20% decrease in FEV₁ (PD₂₀) was calculated by interpolation of the logarithmic dose response curve.

### Statistical Methods

All data from the analyses described in Examples 2-4 were performed with a software package (GraphPad Prism Software, Inc., USA). The significance of differences among groups was assessed by Student's t-test, and analysis of categorical variables was examined by Chi-square test. Pearson's correlation coefficient and linear regression analysis were used to analyze the relationship between the percentage decreases in FEV₁ and Borg score. The PD₂₀ values for ATP and AMP were logarithmically transformed to normalize their distribution and presented as geometric means. All other numerical variables were expressed as the mean±SEM and significance was defined as p<0.05.

### Vagal Activation Experiments in Dogs

The experiments described in Example 5 were performed essentially as described in Pelleg et al. [(1996) J. Physiol. 490(1):265-275] and U.S. Pat. No. 5,874,420, both of which are incorporated herein by reference in their entirety. In brief, they were performed on anesthetized (sodium pentobarbitone, 30 mg kg⁻¹ plus 3 mg kg⁻¹ h⁻¹, intravenous) dogs artificially ventilated with room air using a respirator. Arterial blood pH, P_{O2}, P_{CO2}, body temperature were maintained as described in U.S. Pat. No. 5,874,420. A peripheral vein was cannulated for the administration of physiological saline solution and maintenance doses of anesthetic. Catheters were introduced via the femoral vein and left atrial appendage and positioned in the right atrium and left atrium for the administration of test solutions. The chest was opened by a longitudinal sternotomy. The right cervical vagosympathetic trunk was exposed by a midcervical longitudinal section of the skin and careful dissection of neck muscles and connective tissues. The edges of the cut skin were elevated and secured to create a trough which was filled with warm (about 37° C.) mineral oil. A section of the vagosympathetic trunk was placed on a small plate of black Perspex™ and fine branches were separated from the main bundle by careful dissection using microsurgical tools and a dissecting microscope (Model F212, Jenopik Jena, GmbH, Germany).

Extracellular neural action potentials were recorded using a custom-made bipolar electrode that contained two platinum-iridium wires (1.25 cm×0.0125 cm) connected to a high-impedence first-stage differential amplifier (model AC8331, CW, Inc., Ardmore, Pa.) via a shielded cable. The output of the first-stage amplifier was fed into a second-stage differential amplifier (model BMA-831/C, CWE, Inc.). Isolated fibers were laid on the pair of platinum-iridium wires. Vagal C fibers with chemosensitive endings have a sparse irregular discharge which is not associated with cardiac or respiratory cycles. Confirmation of fiber type was obtained by: first monitoring the response to capsaicin (10 microg kg⁻¹, intra-right atrial bolus); second, monitoring the response to mechanical stimulation of the lungs using gentle probing with forceps as well as inflation of the lungs to 2-3 times the tidal volume; and third, determining the speed of conduction using a stimulating electrode positioned distal to the initial recording site. Nerve fibers with RAR on their pulmonary endings, spontaneously fire a brief (i.e., short lasting) volley of action potentials associated with peak tracheal pressure during each respiratory cycle.

ATP (3 umole kg⁻¹) and capsaicin (10 ug kg⁻¹) were administered as a rapid bolus into the right atrium (5 ml test solution+5 ml physiological saline flush). α,βmATP and β,γmATP were given as one low dose only (0.75 umol kg⁻¹) to avoid systemic side effects. Volume controls consisted of either 5 ml+5 ml or 1 ml+3 ml physiological saline. All injections were performed in the same mode by the same person. To exclude involvement of baroreceptors in the recorded neural activity, the latter was monitored before and after a bolus of nitroglycerine (1 mg; intravenous).

### Example 2

### Airway Responsiveness to AMP and ATP Measured as Change in FEV₁

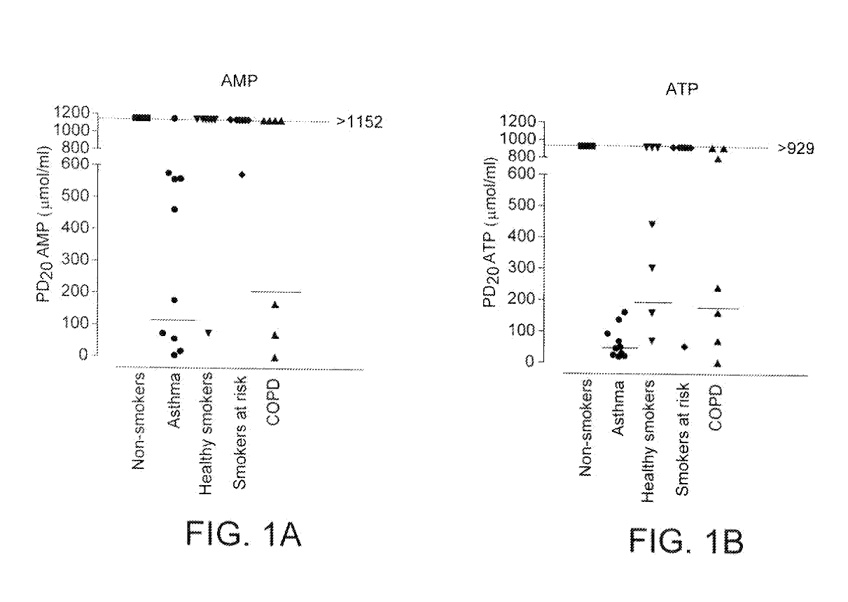

Nineteen of the 40 subjects in the study (47.5%) were responsive to ATP and 13 were responsive to AMP (32.5%). The PD₂₀ geometric means for ATP and AMP were 72.9 (2.9-808.7) umol/ml and 82.9 (0.9-576.0) umol/ml, respectively, in the subjects who had airway responsiveness. The response to either ATP or AMP challenge in healthy non-smokers was negative, i.e., there was either no change in FEV₁ or there was a reduction in FEV₁ of less than 20%. Ten (100%) patients with asthma, four (57%) healthy smokers, one (14%) smoker at risk and four (67%) COPD patients responded to ATP, whereas nine (90%) asthma patients, one (14%) healthy smoker, one (14%) smoker at risk and two (33%) COPD patients responded to AMP (Table 2 and FIG. 1). In the 19 smoking subjects (seven healthy smokers, seven smokers at risk and five COPD), ATP caused bronchoconstriction in twice as many patients as AMP, i.e., 42% and 21%, respectively (p<0.05).

Importantly, while all asthmatic patients responded to low concentrations of ATP, two of the COPD patients failed to respond at even the highest concentration of ATP and the mean PD₂₀ for ATP-responsive COPD patients (178.5 umol/ml) was significantly higher than that for the asthma patients (48.7 umol/ml) (p<0.05) (FIG. 1 and Table 2). These findings provide the basis for a diagnostic test to discriminate between COPD and asthma patients.

The majority of patients (n=5) with COPD were current smokers. While none of the healthy non-smoker control subjects in the study responded to ATP, three of seven healthy smoker control subjects did respond (FIG. 1 and Table 2). Thus, it is probable that the non-smoker COPD patients would be, on average, at least no more, and possibly less, responsive to ATP than smoker COPD patients. Thus, testing for ATP-responsiveness would discriminate asthma patients from both smoker and non-smoker COPD patients.

TABLE 2
PD₂₀ Values Obtained from Individual Patients After Separate Challenge with AMP and ATP

| | Non-smokers | | Asthma | | Healthy | | Smokers | | COPD | |
|---|---|---|---|---|---|---|---|---|---|---|
| | (n = 10) | | (n = 10) | | smokers (n = 7) | | at risk (n = 7) | | (n = 7) | |
| | PD₂₀ | | PD₂₀ | | PD₂₀ | | PD₂₀ | | PD₂₀ | |
| | AMP | ATP | AMP | ATP | AMP | ATP | AMP | ATP | AMP | ATP |
| Subject 1 | >1152 | >929 | 72.0 | 49.0 | >1152 | 159.9 | 576.0 | >929 | >1152 | 808.7 |
| Subject 2 | >1152 | >929 | 576.0 | 44.8 | >1152 | 303.4 | >1152 | >929 | 0.9 | 2.9 |
| Subject 3 | >1152 | >929 | 3.46 | 23.6 | >1152 | >929 | >1152 | >929 | >1152 | >929 |
| Subject 4 | >1152 | >929 | 54.7 | 20.9 | >1152 | >929 | >1152 | >929 | >1152 | >929 |
| Subject 5 | >1152 | >929 | 559.3 | 67.9 | 74.6 | 70.2 | >1152 | >929 | 72.0 | 242.8 |
| Subject 6 | >1152 | >929 | 461.7 | 92.2 | >1152 | 442.4 | >1152 | >929 | >1152 | 72.0 |
| Subject 7 | >1152 | >929 | 176.0 | 160.6 | >1152 | >929 | >1152 | 52.8 | 168.4 | 163.1 |
| Subject 8 | >1152 | >929 | >1152 | 18.1 | | | | | | |
| Subject 9 | >1152 | >929 | 15.8 | 28.1 | | | | | | |
| Subject 10 | >1152 | >929 | 556.4 | 137.0 | | | | | | |
| Geometric | >1152 | >929 | 113.5 | 48.7 | 74.6 | 197.0 | 576.0 | 52.8 | 210.6 | 178.5 |
| mean* | | | | | | | | | | |
| 95% CI | 1152 to 1152 | 929 to 929 | 78.0 to 472.1 | 28.1 to 100.3 | 0 to 0 | −16.1 to 504.1 | 0 to 0 | 0 to 0 | 161.1 to 1224 | 61.7 to 837.6 |

*Geometric mean was calculated by excluding the patients not responding to the highest concentration of AMP (1152 μmol/ml) or ATP (929 μmol/ml). PD₂₀: expressed as μmol/ml; 95% CI: 95% confidence interval.

### Example 3

### Effect of ATP and AMP Challenge on Dyspnoea and Other Symptoms

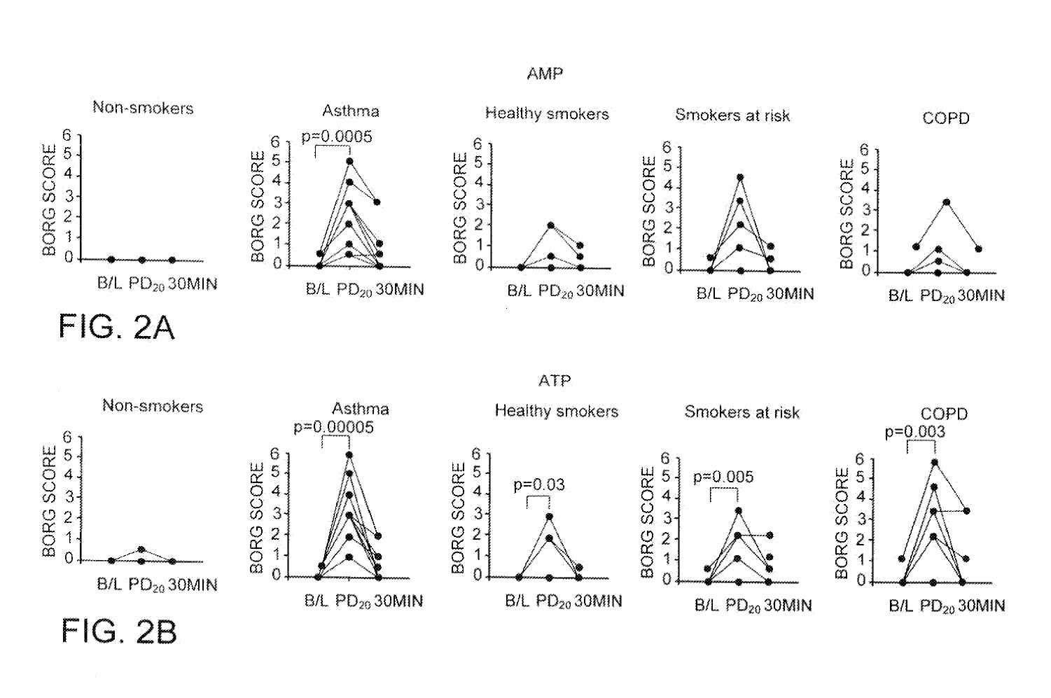

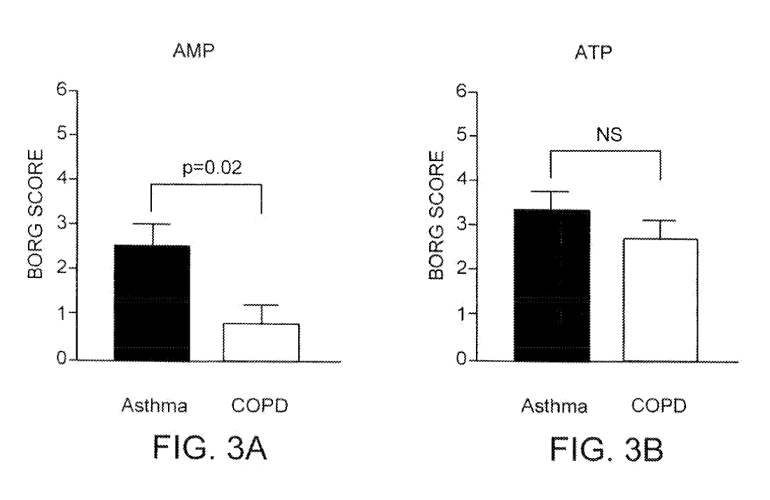

The perception of dyspnoea as assessed by Borg score increased significantly after ATP challenge in asthmatics (from 0.1 to 3.3, p<0.001), healthy smokers (from 0 to 1.3, p<0.03), smokers at risk (from 0.1 to 1.9, p<0.01) and COPD patients (from 0.1 to 2.7, p<0.01) (FIG. 2). In contrast, after AMP challenge, there was a significant increase only in patients with asthma (from 0.2 to 2.5, p<0.001). Borg score after administration of AMP (at PD₂₀) to patients with asthma was higher than in COPD patients (Borg score=2.5 vs. 0.8, respectively, p<0.02), whereas it was similar in the two patient groups after ATP (at PD₂₀) challenge (Borg score=3.3 vs. 2.7, respectively, p>0.05) (FIG. 3).

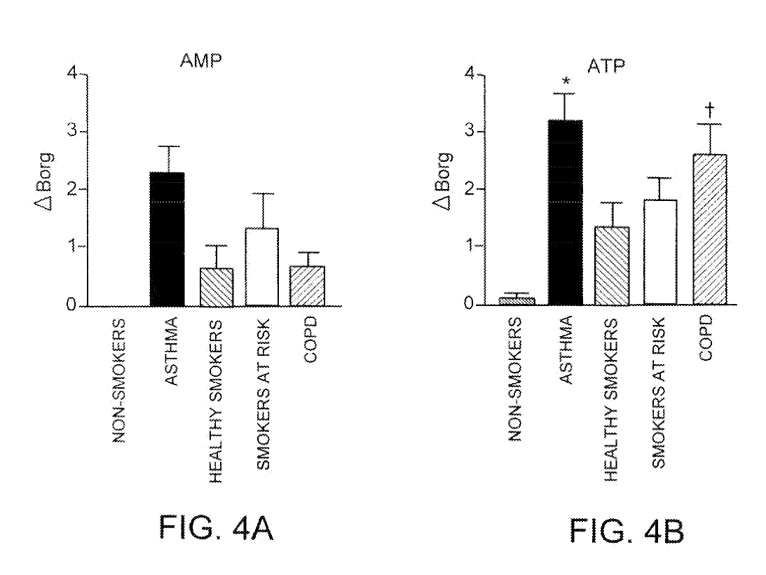

Comparison of the change in Borg score (ΔBorg) after ATP and AMP challenge revealed that ΔBorg was higher after ATP in all groups and this increase was significant after ATP challenge in patients with asthma (ΔBorg_{ATP}=3.2 vs. ΔBorg_{AMP}=2.3, p<0.02) and COPD (ΔBorg_{ATP}=2.6 vs. ΔBorg_{AMP}=0.6, p<0.01) (FIG. 4). There was a negative correlation between the PD₂₀ per se and the Borg score after challenge (at PD₂₀) with both AMP (r=−0.6694, p<0.001) and ATP (r=−0.6521, p<0.001).

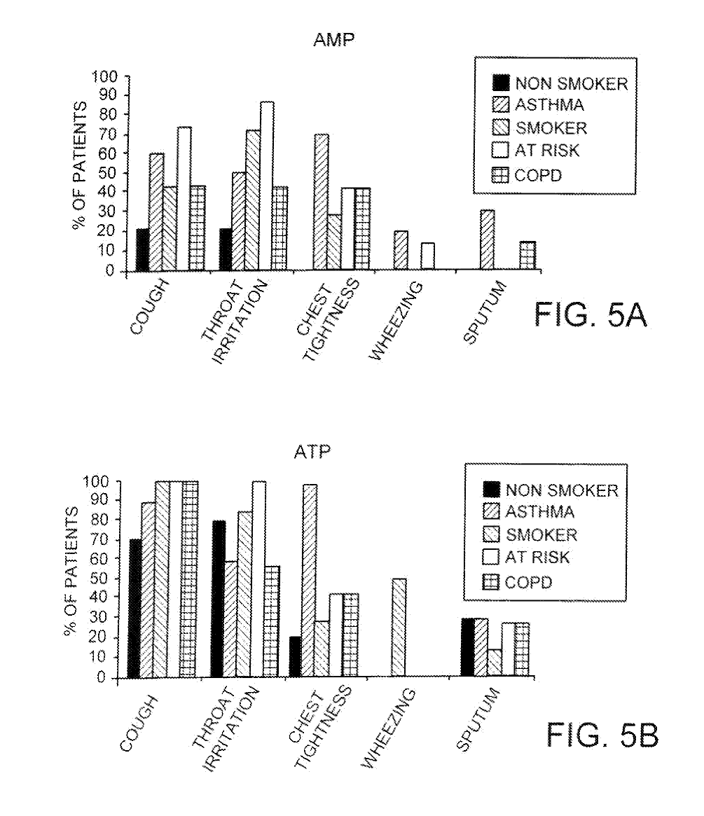

Thirty-six subjects (90%) coughed after ATP challenge whereas AMP challenge caused cough in 19 (48%) subjects (p<0.01). The percentage of subjects who had throat irritation and sputum were also significantly higher after ATP (75% and 28%, respectively) when compared to AMP challenge (53% and 10%, respectively) (p<0.04 for both) (FIG. 5).

When ATP and AMP non-responders or responders were considered separately, the subjects who were non-responsive to ATP had more cough and sputum than AMP non-responders (p<0.01 and p<0.01, respectively). ATP responders also coughed more than AMP responders (p<0.03) and they had more chest tightness and wheezing than ATP non-responders (p<0.001 and p<0.02, respectively). The subjects responsive to AMP reported more chest tightness and sputum than AMP non-responders (p<0.04 and p<0.01, respectively).

### Example 4

### Effect of ATP and AMP Challenge on Airway Caliber

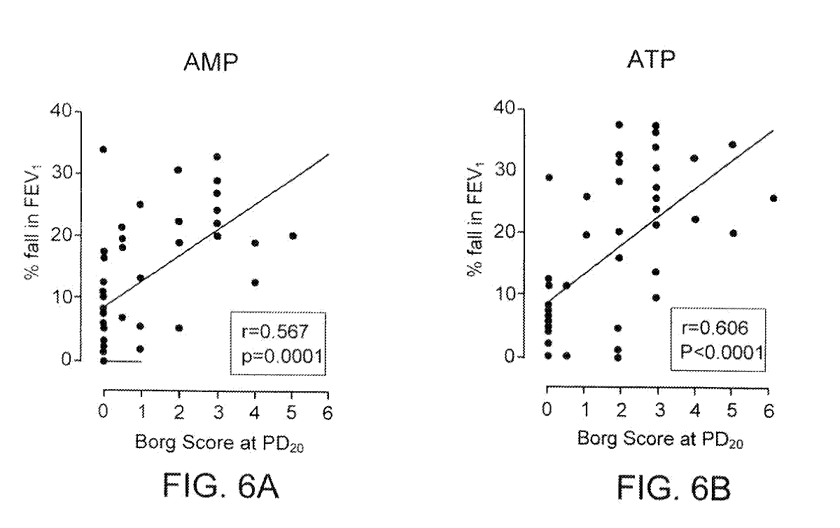

The ATP-induced decrease in FEV₁ expressed as a percentage of the baseline FEV₁ (ΔFEV₁) was greater than that caused by AMP challenge in all groups. This difference was statistically significant in patients with asthma (ΔFEV_{1ATP}=29% vs. ΔFEV_{1AMP}=22%, p<0.03). There was a positive correlation between ΔFEV₁ and Borg score after ATP (r=0.606, p<0.001) and AMP (r=0.567, p<0.01) challenge at PD₂₀ (FIG. 6).

### Example 5

### Extracellular ATP Stimulates RAR-Containing Fibers as Well as C Fibers in Canine Lungs

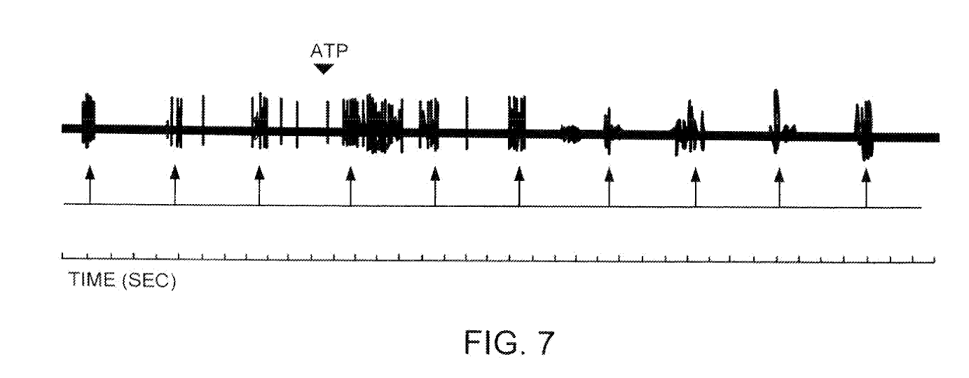

The procedure described in Example 1 was used to measure action potentials in dog pulmonary fibers with RAR on their terminals following administration of various agents. RAR activation generated a brief volley of action potentials associated with each respiratory cycle that was seen before as well as after administration of ATP (FIG. 7). Following the administration of ATP (6 μmol/kg, rapid intravenous bolus) the activity of RAR-containing fibers was prolonged such that the burst of neural action potentials extended into the inter-respiratory cycle interval. Thus ATP modified the activity of the RAR-containing fiber manifested in their transient loss of the rapid adaptability characteristic.

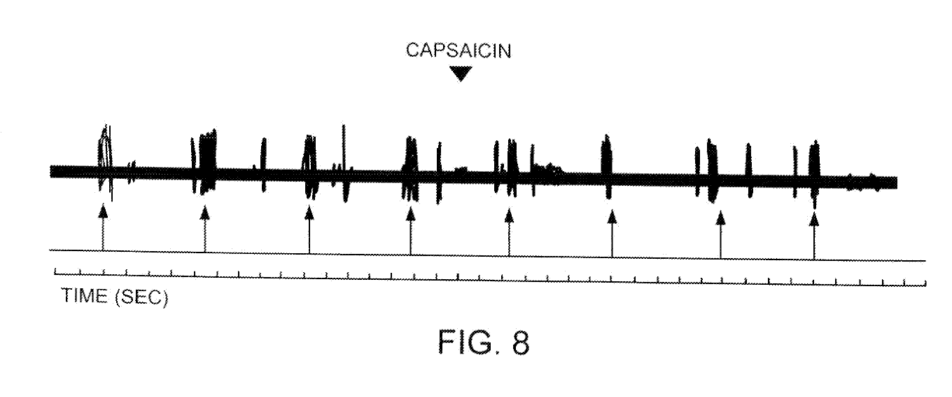

To confirm that the fiber tested was indeed an RAR-containing fiber, a recording of the same preparation was made prior to and following the administration of an intravenous bolus of capsaicin. As can be seen in FIG. 8, capsaicin did not alter the firing pattern of the same RAR-containing fibers that were activated by ATP. This is in agreement with the well-established observations of the inability of capsaicin to stimulate RAR-containing nerve fiber terminals due to the lack of the valinoid receptor (i.e., the capsaicin receptor, VR-1) on these nerve terminals.

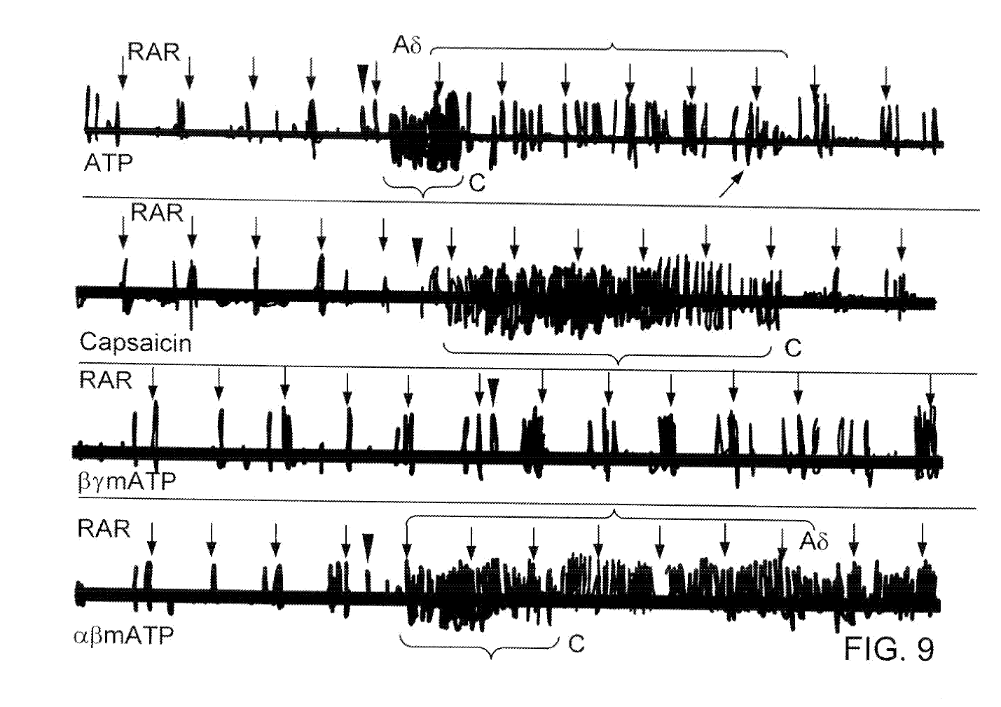

Since capsaicin is known to stimulate C fiber nerve terminals in the lungs, it was of interest to monitor concurrently the response of a C fiber and an RAR-containing fiber to capsaicin, ATP, and analogs of ATP. FIG. 9 is an example of simultaneous recording of the two types of fibers prior to and following the administration of the test compounds. As expected, ATP stimulated both the C fibers and RAR-containing fibers while capsaicin stimulated only the former. In addition, two analogs of ATP (α,βmATP and β,γmATP) that are not readily degraded by ecto-enzymes, acted similarly to ATP; this finding indicated that the action of ATP is not mediated by adenosine the product of its enzymatic degradation.

As can be seen in FIG. 9, α,βmATP was more potent than ATP. This suggested the activation of RAR-containing fibers by ATP and the two analogs is mediated by a particular P2X receptor subtype. Of the seven P2XR, only P2X₁, P2X₃ and heterodimeric P2X_{2/3} are sensitive to α,βmATP [Virginio et al. (1998) Mol. Pharmacol. 53:969-973]. P2X₁ and P2X₃ are rapidly desensitized following stimulation by agonists. However, in the present experiments, repeated administrations of ATP and its similarly active analogs were not associated with desensitization. These findings indicated that: (a) P2X₁ and P2X₃ are at least not the exclusive, or even predominant, receptors involved in the triggering of RAR-containing fibers by ATP; and (b) P2X_{2/3} is at least the predominant, if not exclusive, receptor subtype that mediates this stimulatory action of ATP.

These data indicate how the endogenous compound ATP stimulates pulmonary fibers with RARs on their terminals and thereby could trigger the central cough reflex known to be mediated by these fibers. Thus, ATP activates P2XR (predominantly P2X_{2/3}R) on RAR and thereby triggers a central cough reflex.

### Example 6

### Inhibition of Activation of Pulmonary Vagal Afferent C and A Nerve Fibers by α,βmATP

### Perfused Nerve-Lung Preparations

The method for the extracellular recording of the activity of vagal sensory neurons projecting to guinea pig lungs has been described in detail previously in Canning et al. [(2004) J. Physiol. 557:543-545], which is incorporated herein by reference in its entirety. Briefly, male Hartley guinea pigs (100-200 g) were killed with CO₂ inhalation and exsanguination. The blood from the pulmonary circulation was washed out by in situ perfusion with Krebs' bicarbonate solution (KBS; comprised of 118 mM NaCl, 5.4 mM KCl, 1.0 mM NaH₂PO₄, 1.2 mM MgSO_{4, 1.9} mM CaCl₂, 25.0 mM NaHCO₃, 11.1 mM dextrose, and gassed with 95% O₂-5% CO₂ at pH7.4). The KBS contained 3 uM indomethacin to reduce the indirect influence of tissue prostanoids on sensory fiber activity. Trachea and right lungs, having intact right-side extrinsic vagal innervation by the inclusion, e.g., of right jugular and nodose ganglia, were dissected from the exsanguinated guinea pigs and placed in a two-compartment tissue bath. The right nodose and jugular ganglia, along with the rostral vagus nerve, were placed into one compartment of the tissue bath whereas the lung and trachea were placed into the second compartment of the tissue bath. The two compartments were separately superfused with KBS (6 ml min⁻¹, at 37° C.).

The pulmonary artery and trachea were cannulated with polyethylene (PE) tubing and continuously perfused with KBS (4 ml min⁻¹ and 2 ml min⁻¹, respectively). Prior to the perfusion, 10 punctures were made through the surface of the lung with a 26 gauge needle and thus KBS could exit the lungs via both these puncture ports as well as via the pulmonary veins.

### Discrimination of Single Fiber Activity and Calculation of Conduction Velocities

The recording electrode was manipulated into the nodose ganglion. A mechanosensitive receptive field was identified by bluntly applying a mechanical stimulus (Von Frey hair, 1800-3000 mN) to the lung surface and observing a burst of neural action potentials.

Once a mechanosensitive receptive field was identified, a brief [<1 millisecond (ms)] electrical stimulus was delivered by a small concentric electrode that was positioned over a discrete region of the mechanosensitive receptive field to determine the conduction velocity of the fiber. The receptive field was stimulated electrically with a square pulse (0.5 ms) of increasing voltage (starting at 5 V) until an action potential was evoked. Conduction velocity was calculated by dividing the distance along the nerve pathway by the time between the shock artifact and the action potential evoked by electrical stimulation of the mechanosensitive receptive field.

The response to lung distension was studied by increasing the rate of perfusion through the trachea as described previously [Canning et al. (2004)]. A 2-fold increase in perfusion rate produced about the threshold distending pressure for action potential discharge. To ascertain if particular fiber responded in a slowly or rapidly adapting fashion (i.e., if the particular fiber was a C fiber or an A fiber), the perfusion rate was again doubled, and held for 5-10 sec. An adaptation index of >90% over the initial 5 seconds of the stimulus was considered rapidly adapting and the relevant fibers were considered to be those with RAR on their pulmonary terminals, i.e., A fibers. The nerve fibers with conduction velocities of <1 ms⁻¹ were considered to be C fibers based on previous analyses of the conduction velocity of the vagal compound action potentials, and in accordance with characteristic's of C fibers accepted or known to a person of ordinary skill in the art.

Administration of P2X-Receptor Agonist α,βmATP and P2X₃/P2X_{2/3}-Receptor Antagonist A-317491) to Nerve-Lung Preparations

The P2X-receptor agonist α,βmATP and P2X₃/P2X_{2/3}-receptor antagonist A-31749 were diluted separately in KBS. The preparation was treated sequentially as follows:

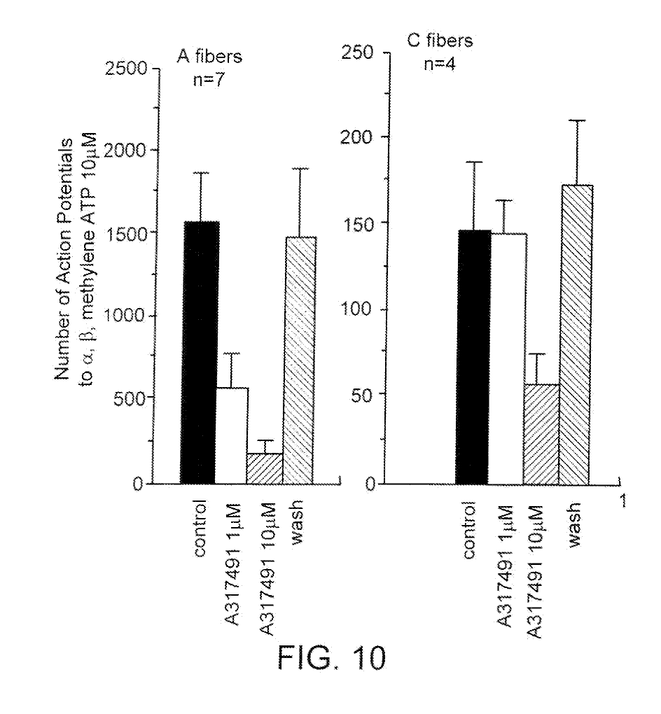

(a) After 30 min of perfusion with KBS, a 1 ml bolus of 10 uM α,βmATP was inoculated into the perfusing solution and the response was recorded (FIG. 10: “control”).
(b) The preparation was perfused for 30 min with KBS containing 1 uM A-31749, a 1 ml bolus of 10 μM α,βmATP was inoculated into the perfusing solution, and the response was recorded (FIG. 10; “A31749 1 uM”).
(c) The preparation was perfused for 30 min with KBS containing 10 uM A-31749, a 1 ml bolus of 10 uM α,βmATP was inoculated into the perfusing solution, and the response was recorded (FIG. 10; “A31749 10 uM”).
(d) The preparation was perfused for 30 min with KBS, a 1 ml bolus of 10 uM α,βmATP was inoculated into the perfusing solution, and the response was recorded (FIG. 10; “wash”).

Both α,βmATP and A-31749 were infused at a rate of 50 ul s⁻¹. α,βmATP and A-317491 were infused through both routes (i.e., via tracheal and pulmonary artery perfusion) in order to increase the consistency with which the they reached the receptive field (lungs) and to reduce the likelihood of “false negative” observations. When Evans blue dye was administered via the trachea alone or the pulmonary artery alone, although it was quickly able to penetrate all tissue compartments regardless of route of administration, in some cases, its distribution was hindered due to obstruction in either the tracheal route or vascular routes.

### Data Analysis

In most of the electrophysiological measurements of afferent neurons that innervate the intrapulmonary airways and lungs, the activity of single neurons were recorded. On rare occasions, where two units were recorded simultaneously, straightforward wave analysis software (TheNerveOflt; PHOCIS, Baltimore, Md., USA) was used to distinguish between the two peaks.

Neuron activity was recorded with a glass microelectrode pulled with a micropipette puller (Sutter Instrument Company P-87, Novato, Calif., USA) and filled with 3 M sodium chloride (resistance ˜2 MΩ). The signal was amplified (Microelectrode AC amplifier 1800; A-M systems, Everett, Wash., USA), filtered (low cut off, 0.3 kHz; high cut off, 1 kHz), displayed on an oscilloscope (TDS 340; Tektronix, Beaverton, Oreg., USA) and chart recorder (TA240; Gould, Valley View, Ohio, USA), and recorded (sampling frequency 33 kHz) into a Macintosh computer for offline analysis (TheNerveOflt; PHOCIS, Baltimore, Md., USA). Tracheal perfusion pressure reflecting the airway smooth muscle contraction was measured with a pressure transducer (P23AA; Statham, Hata Rey, PR, USA) and the pressure was recorded by chart recorder (TA240).

All the activity evoked by a given concentration of agonist was recorded in 1 s bins and analyzed off-line. The response to α,βmATP was deemed to have terminated when the action potential discharge ceased or at such time that the discharge was <2× that observed at baseline. The data are expressed as mean±standard deviation (SD). Student's paired and non-paired t tests were used for statistical analysis and significance was attributed to p<0.05. The n value represents the number of fibers studied; only one fiber was studied per perfused nerve-lung preparation.

### ATP Stimulates Pulmonary Vagal Afferent C and A Fiber Terminals

The action potentials (AP) in C (n=4) and A fibers (n=7) elicited by administration of α,βmATP, a potent selective agonist of P2X₃/P2X_{2/3}-receptors, were quantified as discharge/sec. As can be seen in FIG. 10, the administration of α,βmATP (10 μM, 1 mL bolus), elicited neural AP in nodose C and A fibers terminals of the perfused nerve-lung preparation. α,βmATP induced AP in both types of fibers in a non-desensitizing manner. The frequency of the generated AP was 146±29 in C fibers and 1543±285 in A fibers.

The P2X₃/P2X_{2/3}-Receptor Selective Antagonist A-317491 Inhibits the Activation of Pulmonary Vagal Afferent C and A Fibers by α,βmATP.

The AP in C (n=4) and A fibers (n=7) induced by α,βmATP (10 uM, 1 ml, bolus) in the presence of A-317491 (1 and 10 uM, 30 min) were measured as described above. A-317491 (10 uM) reduced the response of C and A fibers to α,βmATP by 62±5% (p<0.05) and 88±5% (p<0.05), respectively as compared their respective controls. At 1 uM, A-317491 significantly inhibited the action of α,βmATP in A fibers by 59±12%, but had no inhibitory effect on C fibers.

A number of embodiments of the invention have been described. Nevertheless, it will be understood that various modifications may be made without departing from the spirit and scope of the invention. Accordingly, other embodiments are within the scope of the following claims.

1.-15. (canceled)

16. A method of treating an OPD or cough, the method comprising:
(a) identifying a mammalian subject as having an OPD, having symptoms associated with an OPD, or having cough;
(b) administering to the subject a therapeutically effective dose of a pharmaceutical composition that comprises one or more compounds, each compound being of formula (I): 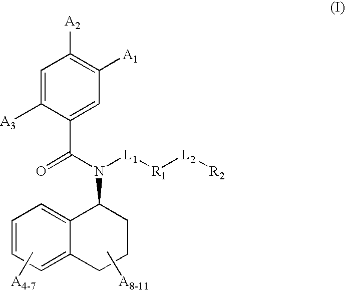
or a pharmaceutically acceptable salt thereof, wherein
A₁ and A₂ are each independently selected from the group consisting of alkoxycarbonyl, alkylcarbonyloxy, carboxy, hydroxy, hydroxyalkyl, (NR_{A} R_{B})carbonyl, —NR_{C}S(O)₂R_{D}, —S(O)₂OH, and tetrazolyl; or
A₁ and A₂ together with the carbon atoms to which they are attached form a five membered heterocycle containing a sulfur atom wherein the five membered heterocycle is optionally substituted with 1 or 2 substituents selected from mercapto and oxo;
A₃ is selected from the group consisting of alkoxycarbonyl, alkylcarbonyloxy, carboxy, hydroxy, hydroxyalkyl, (NR_{A}R_{B})carbonyl, NR_{C}S(O)₂R_{D}, —S(O)₂OH, and tetrazolyl;
A₄, A₅, A₆ and A₇ are each independently selected from the group consisting of hydrogen, alkoxy, alkoxycarbonyl, alkenyl, alkyl, alkylcarbonyl, alkylcarbonyloxy, alkynyl, aryl, carboxy, cyano, haloalkoxy, haloalkyl, halogen, hydroxy, hydroxyalkyl, nitro, —NR_{E}R_{F}, and (NR_{E}R_{F})carbonyl;
A₈, A₉, A₁₀ and A₁₁ are each independently selected from the group consisting of hydrogen, alkoxy, alkoxycarbonyl, alkenyl, alkyl, alkylcarbonyl, alkylcarbonyloxy, alkynyl, aryl, carboxy, haloalkoxy, haloalkyl, halogen, hydroxy, hydroxyalkyl, —NR_{E}R_{F}, (NR_{E}R_{F})carbonyl, and oxo;
R_{A} and R_{B} are each independently selected from the group consisting of hydrogen, alkyl, and cyano;
R_{C} is selected from the group consisting of hydrogen and alkyl;
R_{D} is selected from the group consisting of alkoxy, alkyl, aryl, arylalkoxy, arylalkyl, haloalkoxy, and haloalkyl;
R_{E} and R_{F} are each independently selected from the group consisting of hydrogen, alkyl, alkylcarbonyl, formyl, and hydroxyalkyl;
L₁ is selected from the group consisting of alkenylene, alkylene, alkynylene, —(CH₂)ₘO(CH₂)ₙ—, —(CH₂)ₘS(CH₂)ₙ—, and —(CH₂)ₚC(O)(CH₂)_{q}—, wherein the left end of the group is attached to N and the right end of the group is attached to R₁;
m is an integer 0-10;
n is an integer 0-10;
R₁ is selected from the group consisting of aryl, cycloalkenyl, cycloalkyl, and heterocycle;
L₂ is absent or selected from the group consisting of a covalent bond, alkenylene, alkylene, alkynylene, —(CH₂)ₚO(CH₂)_{q}—, —(CH₂)ₚS(CH₂)_{q}—, —(CH₂)ₚC(O)(CH₂)_{q}—, —(CH₂)ₚC(OH)(CH₂)_{q}—, and —(CH₂)ₚCH═NO(CH₂)_{q}—, wherein the left end of the group is attached to R₁ and the right end of the group is attached to R₂;
p is an integer 0-10;
q is an integer 0-10; and
R₂ is absent or selected from the group consisting of aryl, cycloalkenyl, cycloalkyl, and heterocycle.

17. The method of claim 16, wherein the compound is 5-({(3-phenoxybenzyl)[(1S)-1,2,3,4-tetrahydro-1-naphthalenyl]amino}carbonyl)-1,2,4-benzenetricarboxylic acid (A-317491).

18. The method of claim 16, wherein the OPD is chronic obstructive pulmonary disease (COPD).

19. The method of claim 16, wherein the OPD comprises coughing.

20. The method of claim 16, wherein the OPD is asthma.

21. The method of claim 16, wherein the OPD is selected from the group consisting of acute bronchitis, emphysema, chronic bronchitis, bronchiectasis, cystic fibrosis, and acute asthma.

22. The method of claim 16, wherein the compound has the ability to inhibit a vagal response mediated by a P2R on a vagal afferent nerve terminal.

23. The method of claim 22, wherein the P2R is a P2X receptor.

24. The method of claim 23, wherein the P2X receptor is a P2X₃ receptor.

25. The method of claim 23, wherein the P2X receptor is a P2X_{2/3} receptor.

26. The method of claim 16, wherein the administration of the composition is by intrapulmonary inhalation.

27. The method of claim 16, wherein the administration of the composition is by intravenous bolus injection.

28. A method of inhibiting activation of a P2R on a pulmonary vagal sensory nerve fiber terminal, the method comprising contacting the vagal sensory nerve fiber terminal with one or more compounds, each compound being of formula (I): 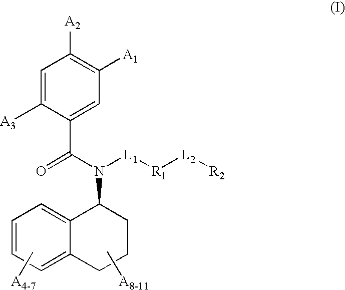
or a pharmaceutically acceptable salt thereof, wherein
A₁ and A₂ are each independently selected from the group consisting of alkoxycarbonyl, alkylcarbonyloxy, carboxy, hydroxy, hydroxyalkyl, (NR_{A} R_{B})carbonyl, —NR_{C}S(O)₂R_{D}, —S(O)₂OH, and tetrazolyl; or
A₁ and A₂ together with the carbon atoms to which they are attached form a five membered heterocycle containing a sulfur atom wherein the five membered heterocycle is optionally substituted with 1 or 2 substituents selected from mercapto and oxo;
A₃ is selected from the group consisting of alkoxycarbonyl, alkylcarbonyloxy, carboxy, hydroxy, hydroxyalkyl, (NR_{A}R_{B})carbonyl, NR_{C}S(O)₂R_{D}, —S(O)₂OH, and tetrazolyl;
A₄, A₅, A₆ and A₇ are each independently selected from the group consisting of hydrogen, alkoxy, alkoxycarbonyl, alkenyl, alkyl, alkylcarbonyl, alkylcarbonyloxy, alkynyl, aryl, carboxy, cyano, haloalkoxy, haloalkyl, halogen, hydroxy, hydroxyalkyl, nitro, —NR_{E}R_{F}, and (NR_{E}R_{F})carbonyl;
A₈, A₉, A₁₀ and A₁₁ are each independently selected from the group consisting of hydrogen, alkoxy, alkoxycarbonyl, alkenyl, alkyl, alkylcarbonyl, alkylcarbonyloxy, alkynyl, aryl, carboxy, haloalkoxy, haloalkyl, halogen, hydroxy, hydroxyalkyl, —NR_{E}R_{F}, (NR_{E}R_{F})carbonyl, and oxo;
R_{A} and R_{B} are each independently selected from the group consisting of hydrogen, alkyl, and cyano;
R_{C} is selected from the group consisting of hydrogen and alkyl;
R_{D} is selected from the group consisting of alkoxy, alkyl, aryl, arylalkoxy, arylalkyl, haloalkoxy, and haloalkyl;
R_{E} and R_{F} are each independently selected from the group consisting of hydrogen, alkyl, alkylcarbonyl, formyl, and hydroxyalkyl;
L₁ is selected from the group consisting of alkenylene, alkylene, alkynylene, —(CH₂)ₘO(CH₂)ₙ—, —(CH₂)ₘS(CH₂)ₙ—, and —(CH₂)ₚC(O)(CH₂)_{q}—, wherein the left end of the group is attached to N and the right end of the group is attached to R₁;
m is an integer 0-10;
n is an integer 0-10;
R₁ is selected from the group consisting of aryl, cycloalkenyl, cycloalkyl, and heterocycle;
L₂ is absent or selected from the group consisting of a covalent bond, alkenylene, alkylene, alkynylene, —(CH₂)ₚO(CH₂)_{q}—, —(CH₂)ₚS(CH₂)_{q}—, —(CH₂)ₚC(O)(CH₂)_{q}—, —(CH₂)ₚC(OH)(CH₂)_{q}—, and —(CH₂)ₚCH═NO(CH₂)_{q}—, wherein the left end of the group is attached to R₁ and the right end of the group is attached to R₂;
p is an integer 0-10;
q is an integer 0-10; and
R₂ is absent or selected from the group consisting of aryl, cycloalkenyl, cycloalkyl, and heterocycle.
